SEQUENCE LISTING (2 sequences)

SEQ ID NO: 1 - DNA, 22 bp, synthetic construct
  source 1..22: mol_type = other DNA
  primer_bind 1..22
gcaaccttgactgccaggaaga

SEQ ID NO: 2 - DNA, 23 bp, synthetic construct
  source 1..23: mol_type = other DNA
  primer_bind 1..23
ctcataacctgcatccttccagc

(19) United States
(12) Patent Application Publication
(10) Pub. No.: US 2026/0284057 A1
(43) Pub. Date: Sep. 24, 2026

(54) FOLIC ACID SUPPLEMENTATION FOR TREATMENT OF FABRY DISEASE

(71) Applicant: The Research Foundation for Mental Hygiene, Inc., Menands, NY (US)

(72) Inventors: Mohammed A. Junaid, Monmouth Junction, NJ (US); Sabiha Khatoon, Staten Island, NY (US)

(73) Assignee: The Research Foundation for Mental Hygiene, Inc., Menands, NY (US)

(21) Appl. No.: 19/568,058

(22) Filed: Mar. 16, 2026

Related U.S. Application Data
(60) Provisional application No. 63/774,855, filed on Mar. 20, 2025.

(51) Int. Cl. A61K 31/519; A61K 31/445; A61K 38/47; A61P 3/00

(52) U.S. Cl. CPC A61K 31/519; A61K 31/445; A61K 38/47; A61P 3/00; C12Y 302/01022

(57) ABSTRACT

A therapeutic approach for treating patients with Fabry disease by supplementing or co-administration of synthetic folic acid (FA) to substantially stimulate α-galactosidase (AGLA) activity in combination with another therapy. Stimulation of AGLA activity may help Fabry disease patients on different types of primary therapeutic regiments manage clinical symptoms with a reduced dose of the primary treatment medication. Folic acid may thus be co-administered with an enzyme replacement therapy, such as recombinant α-galactosidase A, or a chaperone therapy, such as specific inhibitor, 1-deoxygalactonojiromycin, to improve the efficacy of the treatment.

### CROSS-REFERENCE TO RELATED APPLICATIONS

The present application claims priority to U.S. Provisional Application No. 63/774,855, filed on Mar. 20, 2025, hereby incorporated by reference in its entirety.

### INCORPORATION BY REFERENCE

The XML plain text file entitled 151P031.xml dated Mar. 10, 2026 and having a size of 3,090 bytes containing a Sequence Listing is hereby incorporated by reference.

### BACKGROUND OF THE INVENTION

### 1. Field of the Invention

The present disclosure relates to treatments for Fabry disease and, more particularly, to the use of folic acid supplementation to stimulate «-galactosidase activity in the Fabry disease.

### 2. Description of the Related Art

Fabry disease (FD) is a rare inherited disorder with severe lifelong manifestations due to accumulating glycosphingolipids in several tissues and organs. FD is one of the lysosomal storage disorders, which is marked by a severe deficiency of the lysosomal catabolic enzyme α-galactosidase (AGLA) EC: 3.2.1.22 arising from over 400 different mutations identified in the GLA gene so far. This gene is located on the long arm of X-chromosome (chrX: 101,393,273-101,408,012). Accumulation of specific lipids in affected individuals begin at birth, and continues throughout the lifespan, resulting in diverse signs and symptoms associated with FD. Reduced life expectancy of males and females of 58.2 and 74.7 years respectively has been reported for people suffering from FD. General prevalence of FD is thought to be 1 in 40000 to 117000 worldwide, however, recent estimates based on largescale screening of neonates indicated prevalence of variants forms of FD to be as high as 1 in 3700 males in certain populations. While genetic in nature, FD afflicts all racial and ethnic groups across the globe and is not concentrated in any specific community. Because the FD gene is located on X-chromosome and follows X-linked inheritance pattern, it often goes undetected in female population or is missed entirely. There are two different types of FD recognized based upon the age of onset of the symptoms—a classic early onset type displaying symptoms as early as two years of age, and a variant late onset type with symptoms unnoticed sometimes until third decade of life.

The gene GLA codes for the enzyme AGLA that acts as a homodimeric protein in lysosomes, where it catabolizes the hydrolysis of the terminal «-galactosyl residue from various glycospingolipids. Due to lack of the AGLA activity in FD, specific glycospingolipids, such as globotriaosylceramide (Gb3) accumulates in various cell types leading to diverse clinical manifestation of the FD. Primary symptoms of FD include renal insufficiency leading to kidney failure, cardiac involvement causing premature myocardial infarction and CNS pathology often leading to early stroke. Without proper treatment, invariably FD results in early death between the fourth and the fifth decade of an affected individual's life.

The majority of GLA mutations are missense, affecting the structural integrity of the AGLA protein, and consequently the enzyme activity. Residual enzyme activities with certain missense mutations are well correlated with the severity of the clinical symptoms. To alleviate associated symptoms and delay disease progression, several different promising treatments approved by FDA are available for FD patients depending on the type of mutation present in the GLA. Foremost being enzyme replacement therapy that involves a recombinant AGLA infused intravenously every two weeks to affected individuals. This form of treatment is indicated for individuals with all types of mutations resulting in a lack of or severely diminished AGLA activity. Fabrazyme (AGLAsidase beta) and Elfabrio (pegunigalsidase alfa) are the two approved enzyme replacement medications available in the market. The other form of therapy is a small molecule oral chaperone therapy, that involves repairing a mutant AGLA protein that has misfolded because of missense mutations. A specific AGLA enzyme inhibitor, Migalastat also known as Galafold (chemical name: 1-deoxygalactonojiromycin) allows for a misfolded protein that is retained in the endoplasmic reticulum to undergo proper folding thereby regaining substantial AGLA activity in certain affected individuals. However, this therapy will not work for everyone and is restricted to those who have a missense mutation leading to retaining of misfolded protein in the endoplasmic reticulum. These treatment strategies suggest that even any incremental increase in AGLA will substantially alter severity of disease progression allowing for improvement in the quality of life in affected individuals.

Accordingly, there is a need in the art for a therapeutic approach to treating Fabry disease that addresses the deficiency in AGLA activity.

### BRIEF SUMMARY OF THE INVENTION

The present invention provides a therapeutic approach for treating Fabry disease by supplementing with synthetic folic acid (FA) to substantially stimulate «-galactosidase (AGLA) activity. Such stimulation of AGLA activity may help FD patients on different types of therapeutic regiments for managing the clinical symptoms by way of reduced dose of the treatment medication.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a graph showing the stimulation of α-galactosidase A (AGLA) in lymphoblastoid cells with various concentrations of folic acid (FA). GM7027 and GM7053 cell lines were from male and female individuals respectively.

FIG. 2 is a graph and image showing that FA supplementation stimulates AGLA expression in lymphoblastoid cells at transcript and protein levels. (A) Transcript levels were measured by RT-PCR using primer pair HP200154 for GLA and HP200179 for HPRT1. Values are median of three independent experiments and bars show the SEM. (B) Western blot analysis of lymphoblastoid cells exposed to FA concentrations, (25 μg protein each) were probed with an anti-AGLA rabbit monoclonal antibody. (C) The densities of the bands in (B) were calculated by using ImageJ software for quantification. Picture of a representative blot.

FIG. 3 is a graph of the stimulation of recombinant AGLA by FA.

FIG. 4 is a pair of graphs showing the kinetic properties of recombinant AGLA in the presence of FA.

FIG. 5 is a pair of graphs showing that FA reverses DGJ induced inhibition of recombinant AGLA activity.

FIG. 6 is graph showing that FA stimulates 1-deoxygalactonojiromycin (DGJ) mediated rescue of AGLA activity in lymphoblastoid cells from a FD individual.

### DETAILED DESCRIPTION OF THE INVENTION

Referring to the figures, wherein like numerals refer to like parts throughout, the present invention is a therapeutic approach for treating Fabry disease by supplementing with synthetic folic acid (FA) to substantially stimulate «-galactosidase (AGLA) activity. Synthetic folic acid (FA) supplementation has been shown to substantially stimulate AGLA activity in human lymphoblastoid cells, as well as of pure recombinant human enzyme, in a concentration dependent manner. Such stimulation of AGLA activity may therefore help FD patients on different types of therapeutic regiments for managing the clinical symptoms by way of reduced dose of the treatment medication. In addition, the small molecule chaperone mediated rescue of AGLA in a patient's cell line, is further stimulated by FA, again in a concentration dependent manner. The present invention is therefore beneficial for Fabry disease individuals and invention may be used in combination with enzyme replacement therapy, such a Fabrazyme or Elfabrio, or with a specific chaperone therapy, such as Galafold. As an example, the administration of 0.4 to 4 milligrams of FA per day via oral administration may achieve the effects of the present invention. This dosage is consistent with the recommended daily dosage for general population and women with a prior pregnancy of a child with neural tube defect and would achieve the amount of folic acid falls tested in the current application (15-150 ng/ml).

### Example

### Materials and Methods

4-methylumbelliferone (4-MU), 4-methylumbelliferyl-α-D-galactopyranoside (4-MU-α-Gal), FA, and routine chemicals were purchased from Sigma-Aldrich (St. Louis, MO). RPMI 1640 media, α-galactosidase A rabbit recombinant monoclonal antibody (MA5-35650) and Falcon tissue culture dishes were purchased from ThermoFisher Scientific (Grand Island, NY). Fetal bovine serum, and heat inactivated, dialyzed fetal bovine serum (catalog #S12810H) were bought from Atlanta Biologicals (Flowery Branch, GA). Recombinant α-galactosidase A bearing human sequence representing residues 1-429 (Accession #P06280) with a C-terminal 6-His tag, >95% purity was purchased from R&D Systems (Minneapolis, MN). RT-PCR primer pairs HP200154 for GLA (NM_000169), HP204660 for beta actin (NM_001101), HP200179 for HPRT1 (NM_000194) were purchased from Origene Rockville, MD.

### Cell Culture

Lymphoblastoid cells were obtained from the Coriell Institute for Medical Research, Camden, NJ. Normal male and female lymphoblastoid cell lines GM7027 and GM7053, respectively, belonged to reference CEPH/UTAH Pedigree 1340. Cell line GM 04391 also used in the current study was established from an individual diagnosed with FD. These cells are available to researchers from Coriell Institute for Medical Research, and the current study was conducted under the IRB protocol number NK12025-71. These cell lines were routinely grown in RPMI 1640 medium (catalog #21870-084), supplemented with 10% fetal bovine serum, 2 mM glutamine, 1 mM sodium pyruvate and antibiotics (penicillin and streptomycin, 100 μg/ml each). Prior to exposure to FA, cells were grown for 72 hours in RPMI 1640 FA free media (ThermoFisher Scientific, catalog #27016-021) containing 10% dialyzed and heat inactivated fetal bovine serum, 2 mM glutamine, 1 mM sodium pyruvate and antibiotics (penicillin and streptomycin, 100 μg/ml each). The same media (8 ml) was supplemented with FA at concentrations ranging from 0-500 ng/ml medium in 25 cm² Nunc EasYFlask™ cell culture flasks at about a density of 8×10⁶ cells in 8 ml media. Cells were grown in a humidified incubator maintained at 37° C. in an atmosphere comprising of 5% CO₂ in air. After three days of exposure to FA, cells were collected by centrifugation (800×g for 5 min), rinsed twice with ice-cold normal saline, and stored frozen at −80° C. until further processed.

### Extraction of Nucleic Acids

DNA, RNA and proteins were extracted from frozen lymphoblastoid cells by Qiagen AllPrep DNA/RNA/Protein isolation kit according to manufacturer's stated procedure. Amounts and purity of DNA and RNA eluted were determined by SmartDrop by Accuris Edison, NJ. RNA was stored at −80° C., whereas DNA/protein were stored at −20° C. until further used. cDNA synthesis was performed with 100 ng RNA using Qiagen first strand cDNA kit as described earlier.

### Cytosine Methylation

Targeted region of interest (ROI) bisulfite sequencing was performed for a 573 bp fragment of GLA gene comprising of nucleotides chrX: 101407790-101408363. This fragment comprises of 34 CpG sites in a single CpG island.

### Cell Free Extracts

Cell pellets were resuspended in 200 μl of 25 mM citrate-phosphate buffer pH 4.6 and extracts were prepared with Fisherbrand Sonifier FB505 (ThermoFisher Scientific) equipped with cup horn as described earlier [18]). Clear supernatant obtained after centrifugation at 14,500 rpm for 10 minutes was stored frozen at −20° C. until used. Protein concentrations were measured by the modified Folin phenol method using BSA as the standard.

### Cell Free Extracts

Cell pellets were resuspended in 200 μl of 25 mM citrate-phosphate buffer pH 4.6 and cell free extracts were prepared using a Fisherbrand Sonifier FB505 (ThermoFisher Scientific) equipped with cup horn as described herein. Clear supernatant obtained after centrifugation at 14,500 rpm for 10 minutes at 4° C. was stored frozen at −20° C. until further used. Protein concentrations were measured by the modified Folin phenol method using BSA as the standard.

### Protein Estimations

Protein concentrations were measured by the modified Folin phenol method using bovine serum albumin as the standard such as that disclosed in O·H. Lowry, N.J. Rosebrough, A. L. Farr, R. J. Randall, Protein measurement with the Folin phenol reagent, J. Biol. Chem. 193 (1) (1951) 265-275].

### AGLA Activity

AGLA was measured in 96-well plates as described [20, 21]. Briefly, 50 μl of extract in 25 mM citrate-phosphate buffer pH 4.6 was incubated with 50 μl of the substrate (2 mM 4-MU-α-Gal dissolved in citrate-phosphate buffer, pH 4.6)) for up to 3 hours at 37° C. under shaking conditions. Reaction was inhibited by mixing with 100 μl of 0.1 M glycine-carbonate buffer, pH 11. Product formation was measured in Spectramax M3 plate reader (Molecular Devices, San Jose, CA) using excitation and emission wavelengths 365 nm and 450 nm, respectively. Florescence readings were converted to molarities using standard curve generated with defined concentrations of 4-MU in same chemical composition. A master mix with cell free extract was prepared when incubations were made with the FA.

### Western Blotting

Western blot analysis was done as described herein. Non-specific sites on the membranes were blocked for two hours with 5% nonfat dry milk in TBST, incubated with primary antibody (1:250) overnight at 4° C., then incubated with horse radish peroxidase-conjugated secondary antibodies (1:2,000) at room temperature for two hours, and developed by using the SuperSignal West Pico chemiluminescent substrate (ThermoFisher, Rockford, IL). Images were captured on an iBright FL1500 Imaging System (ThermoFisher, Rockford, IL).

### Statistics

Each experiment was performed in triplicates. Values are presented as means±SEM, and results are considered significant when P<0.05.

### Results

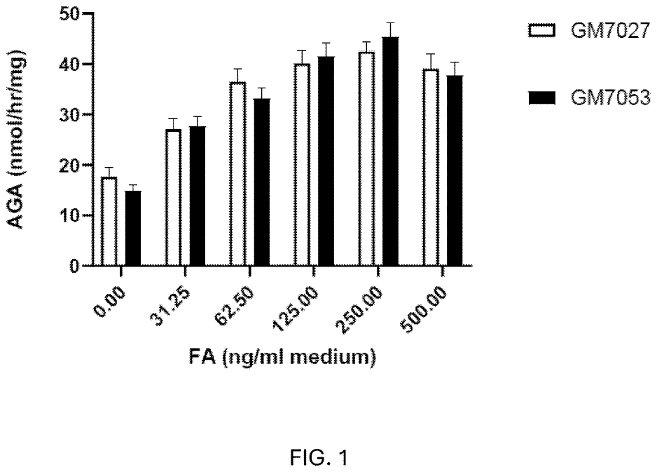

When lymphoblastoid cells in culture conditions are exposed to FA supplementation, it causes significant stimulation of lysosomal AGLA transcript, protein, and enzyme activity. This increase in AGLA in lymphoblastoid cells in culture by FA supplementation increased in a concentration dependent manner. Enzyme activity measurements in cell extracts displayed that AGLA activity in lymphoblastoid cells in culture is increased significantly with as low FA concentration as 31.25 ng/ml medium, as seen in FIG. 1. Referring to FIG. 1, cell lines GM7027 (male) and GM7053 (female) were grown in FA free media for three days prior to incubating with the indicated FA concentrations in RPMI 1640 medium. After three days, cells were collected by centrifugation, rinsed with normal saline, and AGLA activity determined. Values are median of three independent experiments and bars show SEM. AGLA values at all FA concentrations were significantly different (P<0.05) from those at zero concentration.

Increased AGLA enzyme activity was measurable with highest FA concentration tested at 250 ng/ml medium. At further higher concentrations, FA mediated stimulation begins to abolish, and in fact it causes the AGLA activity to inhibit. The stimulation of AGLA activity by FA was unaffected irrespective of whether the cells were derived from a male or a female subject. The increase in AGLA activity in both genders was found to be of similar extent.

The FA mediated increase in AGLA activity seen in lymphoblastoid cells is specific since enzyme activities of several other lysosomal enzymes tested remained unchanged upon incubation with similar FA concentrations. Enzyme activities of aryl sulfatase A (EC: 3.1.6.8), aryl sulfatase B (EC: 1.6.12), α-D-glucosidase (EC: 3.2.1.20), β-glucoronidase (EC: 3.2.1.31), β-D-galactosidase (EC: 3.2.1.23), α-D-mannosidase (EC: 3.2.1.24), α-L-fucosidase (EC: 3.2.1.51), in lymphoblastoid cells upon FA supplementation for 4-days at all concentrations were unaffected, as seen in Table 1 below.

TABLE 1
Lysosomal enzymes tested for effect of folic acid supplementation

| | Enzyme |
|---|---|
| | Commission |
| Enzyme | Number |
| Aryl sulfatase A | (EC: 3.1.6.8) |
| Aryl sulfatase B | (EC: 3.1.6.12) |
| α-L-fucosidase | (EC: 3.2.1.51) |
| β-D-galactosidase | (EC: 3.2.1.23) |
| β-glucoronidase | (EC: 3.2.1.31) |
| α-D-glucosidase | (EC: 3.2.1.20) |
| α-D-mannosidase | (EC: 3.2.1.24) |
| Tripeptidyl peptidase I | (EC: 3.4.14.9) |

Following lysosomal enzymes activities were tested that remained unchanged in lymphoblastoid cells with various FA concentrations in RPMI 1640 medium. Each of the enzyme's activity was tested with published procedures.

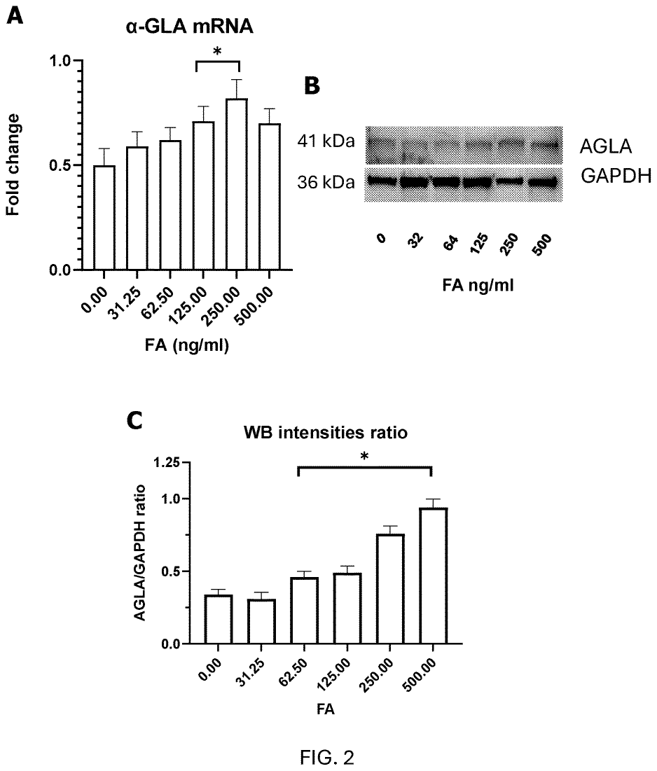

Quantitative RT-PCR data using Origene primer set HP200154 confirmed increased expression of GLA transcript in lymphoblastoid cells supplemented with FA (FIG. 2). This validated primer set utilized forward 5′ GCAACCTTGACTGCCAGGAAGA 3′ (SEQ ID NO: 1) and reverse 5′ CTCATAACCTGCATCCTTCCAGC 3′ (SEQ ID NO: 2) primers, respectively, which amplifies a 107 bp fragment from the cDNA. Transcript levels at zero FA were compared with those at indicated concentrations. AGLA transcript values at FA concentrations 125 ng/ml and higher were significantly different (P<0.05) from those at zero concentration, while those at lower concentrations also had higher expression tendencies but were not statistically significant.

Referring to FIG. 2, cells were grown in FA free medium for three days prior to incubating with the indicated FA concentrations in RPMI 1640 medium. After three days, cells were collected by centrifugation, rinsed with normal saline, and RNA or proteins were prepared. Western blot analysis using an anti-AGLA rabbit polyclonal antibody displayed that AGLA protein expression increased with increasing FA concentration in the medium. There is a basal level of expression of AGLA even in the absence of FA, and thereafter the AGLA expression increased in a concentration dependent manner with increasing FA concentration FIG. 2. The lowest concentration tested was 31.25 ng FA/ml of RPMI 1640 medium.

It was next tested whether enzyme activity of the purified GLA protein is also modulated with FA incubation. Recombinant human alpha-GLA protein with a C-terminal 6-His tag was used, expressed in CHO cell line (RND systems, 6146-GH) as the source of purified enzyme.

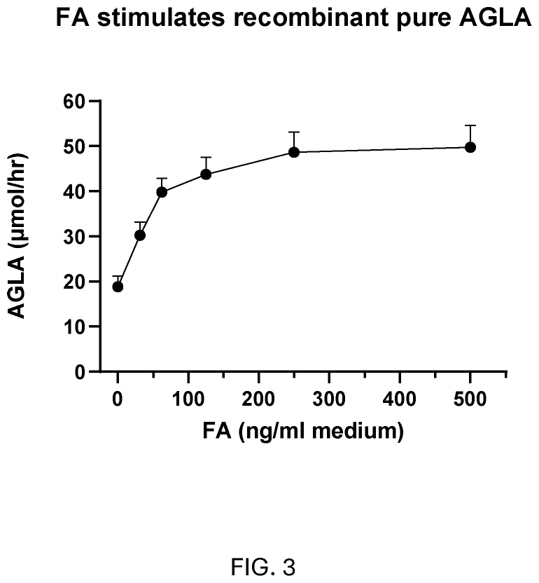

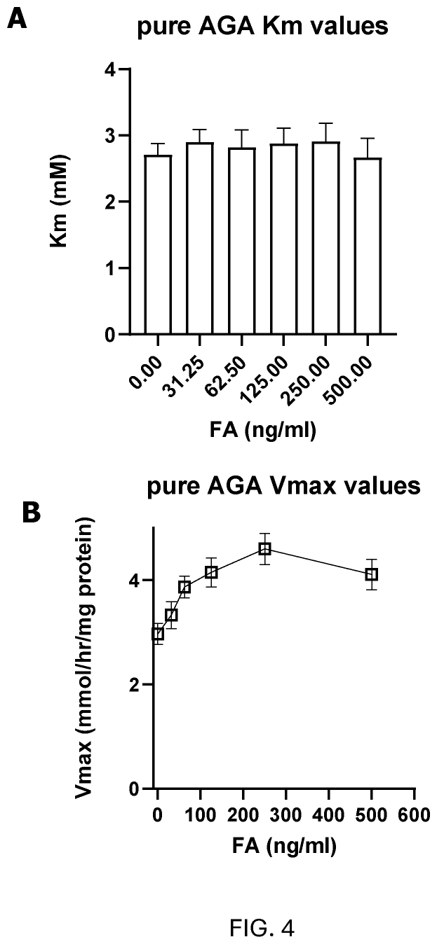

Referring to FIG. 3, recombinant AGLA (Accession #P06280) activity was determined in the presence of indicated FA concentration using 4-MU-α-Gal as the substrate. Release of 4-MU was determined fluorometrically. Values represent means with bars show SEM for three independent measurements. AGLA activity was statistically significant (P<0.05) at all concentrations when measured against zero FA. As shown in FIG. 3, AGLA activity is stimulated by in vitro incubation with FA, again in a concentration dependent manner showing a similar trend as lymphoblastoid cells with relation to FA concentrations. Kinetic parameters evaluated of the AGLA activity at various concentrations of FA demonstrated that the stimulation of the enzyme activity is of mixed type with increasing Vmax but unchanged Km (FIG. 4). Km was not statistically significant between zero and various FA concentrations, whereas Vmax at FA higher than 62 ng/ml was significantly different (P<0.05). Vmax at 31.25 ng/ml FA showed upward trend but was not statistically significant.

Referring to FIG. 4, AGLA activity was determined at various 4-MU-α-Gal concentrations at indicated FA concentration. Km (A) and Vmax (B) were determined from double reciprocal Lineweaver-Burk plots. Values are medians and bars show SEM of three independent measurements.

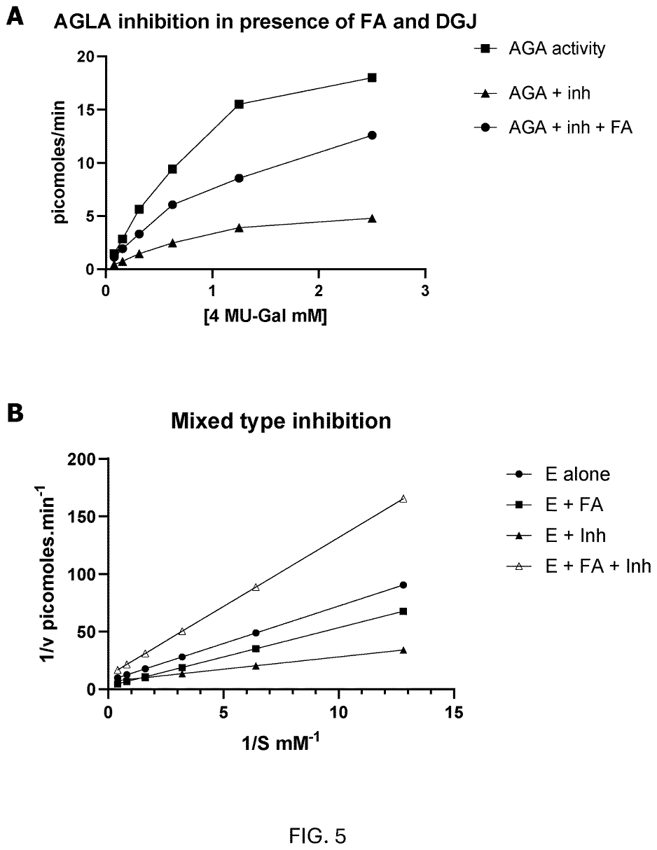

Whether GLA activity inhibited by specific inhibitor, deoxygalactonojirimycin (Migalastat or Galafold), is restored by FA incubation was tested next. As shown in FIG. 5, FA incubation of GLA activity inhibited by deoxygalactonoirimycin is significantly restored by FA by over 50% (FIG. 5). The order of preincubation of GLA with either FA or the inhibitor displayed interesting results. When GLA was preincubated with the inhibitor followed by addition of FA restored the GLA activity by about 50%. Preincubation of GLA with FA for 15 minutes on ice, followed by addition of the inhibitor offered additional protection of GLA activity from being inhibited by the inhibitor, as seen in Table 2 below.

TABLE 2
FA preincubation with AGLA is beneficial)

| Condition | pmol · Min⁻¹ |
|---|---|
| Enzyme alone | 9.42 ± 0.4 |
| Enzyme + DGJ (7 μM) | 2.47 ± 0.2* |
| Enzyme + DGJ + FA | 6.06 ± 0.5* |
| Enzyme + FA + DGJ | 6.96 ± 0.5* |

Recombinant AGLA was incubated with or without DGJ at 7 μM. AGLA activity was measured by incubating with FA that was added either along with DGJ, or 15 minutes prior to adding DGJ on ice. Values represent mean±SEM of three independent experiments and * denotes significantly different from activity without DGJ (P<0.05).

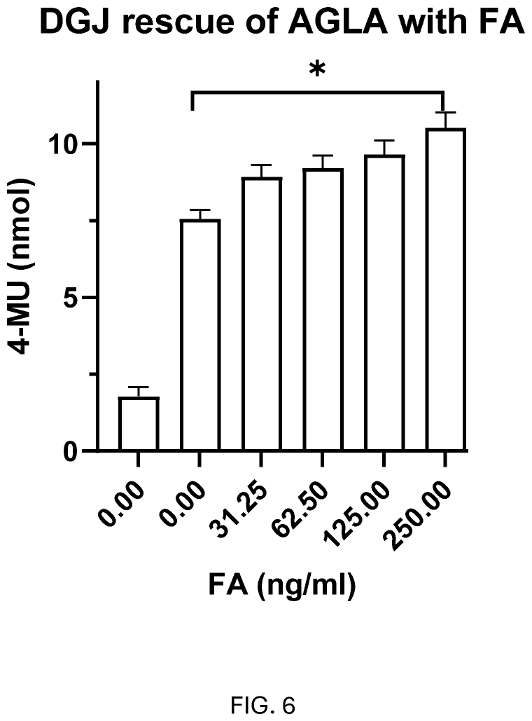

Whether AGLA activity restored by small molecule chaperone DGJ therapy compound shows any effect upon incubation with FA was also tested. Referring to FIG. 6, FA stimulates DGJ mediated rescue of AGLA activity in lymphoblastoid cells from a FD individual. AGLA activity in the cell line GM04391 established from a FD individual was measured without or with DGJ (7 μM), at indicated concentrations of FA in the media. Values represent mean±SEM of three independent experiments and *denotes significantly different from activity without DGJ (P<0.05). As seen in FIG. 6, the AGLA activity in lymphoblastoid cells from an individual with FD was low. After incubation with 7 μM concentration of DGJ, the AGLA activity was increased by >4-folds compared to cells incubated without DGJ. This AGLA activity restored by DGJ in the LCL, was further stimulated by incubation with FA again in a concentration dependent manner, with the FA even at 31.25 ng/ml level stimulating AGLA activity by 18%. The restored AGLA activity continues to increase further with increasing FA concentration of 250 ng/ml.

Targeted methylation sequence was then performed of one CpG island (474 bp) in GLA gene near the promoter region to see if FA mediated hypermethylation of cytosine residues. The results indicated that none of the 34 CpG sites analyzed within this 474 bp fragment had higher methylation rate compared to cells incubated without FA. Methylation ratios across all cytosines within the 34 CpG sites remained unchanged (data not shown).

Together, the data from this study clearly demonstrates that FA supplementation increases AGLA mRNA and protein expression in the lymphoblastoid cells and further, the purified enzyme activity is also stimulated by FA incubation, in concentration dependent manner. Additionally, we provide convincing proof that AGLA activity restored by small molecule chaperone DGJ therapy is further augmented by incubation with increasing FA supplementation.

### Discussion

FD is a debilitating inherited disability afflicting people of all ethnicities around the globe. The introduction of promising therapies in the form of enzyme replacement and chaperone therapies have alleviated suffering individuals' difficulties and contributed significantly to improved lifestyles. Successes of both therapies demonstrated that any incremental increase in the AGLA activity in affected individuals is of paramount importance in lessening the symptoms severity. The present invention demonstrates that both types of therapies may greatly benefit if FA supplementation is simultaneously included with these treatment modalities.

While screening beneficial and potentially detrimental epigenetic effects of the vitamin FA, we observed potential beneficial effects of FA supplementation through substantially augmenting the AGLA activity in human lymphoblastoid cells and the pure recombinant enzyme. Mandatory synthetic FA supplementations of breakfast cereals and grains was instituted to prevent neural tube defects (NTDs) in newborns. This single dietary modification is credited with over 70% reduction in incidences of NTDs. Thus, there is widespread use of FA supplementation in the general population. The exact molecular mechanism involved in FA preventing NTDs are unknown, however, FA is cofactor in enzymes that participate in one-carbon metabolism, and it is expected that combinations of such metabolic activities apart from stimulated nucleic acid synthesis may be responsible.

The present invention demonstrates that FA supplementation stimulates AGLA through two separate but interconnected mechanisms. First, FA supplementation of lymphoblastoid cells in culture stimulated AGLA transcript and protein expression. This is happening at the level of transcription, which may be consequent to epigenetic effects of FA. However, none of the cytosine residues that were tested were found within a single CpG island showing any differential methylation status under FA supplementation to support that this particular CpG island participates in any direct epigenetic effect. The 34 CpG sites that were tested were in one CpG island are in the vicinity of the gene promoter. This suggests that FA is not exerting any direct epigenetic effect on the one CpG island tested, which argues that additional CpG sites may be present in the GLA gene, or FA may promote augmented AGLA expression through another yet unidentified gene that may regulate the GLA gene expression.

Second, in vitro FA incubation also increases AGLA activity in a concentration dependent manner, which is reflected by stimulation of pure recombinant AGLA protein. Kinetic measurement indicated increase in Vmax but not Km of the enzyme. The binding sites for FA and substrates are expected to be separate since enzyme activity stimulation was found to be of mixed type. Thus, FA is also able to stimulate the AGLA enzyme activity as a direct modulator. In the past, presence of glucose, melibiose and raffinose sugars were shown to stimulate AGLA activity, while trehalose treatment induced a marginal increase. Thus, FA seems to be a more potent stimulator of AGLA activity both under in vivo and in vitro conditions.

Irrespective of the gender, the increase in AGLA activity was comparable in both male and female cell lines with FA concentrations. This is important since the GLA gene is localized on X-chromosome, and women do exhibit X-linked inheritance issues for GLA expression due to which they often experience delayed diagnosis for FD.

The FA mediated stimulation of AGLA was found to be highly specific for this lysosomal enzyme, since no such stimulation was observed with several other lysosomal enzymes tested. Of importance is beta-galactosidase 1 that acts on substrate showing only a change in glycosidic linkage orientation in comparison to AGLA. Such specificity is expected, since both AGLA and beta-galactosidase 1 are separate proteins encoded by different genes. Thus, structurally, there may be motifs in AGLA that are amenable to binding FA and consequently stimulating the enzyme activity, and such motifs may be lacking in other lysosomal enzymes we tested. In a similar manner, DGJ specifically inhibits the AGLA activity while no such inhibition is observed with beta-galactosidase 1. Earlier reports had found that AGLA activity is stimulated by ammonium chloride possibly through change in microenvironment at the lysosomal level. Sphingolipid activator protein B (saposin B) stimulates the degradation of the accumulated material globotriaosylceramide, presumably through micellar structure formation and recognition of the same by AGLA. Quinovose (6-deoxy-D-glucose) was specifically shown to stimulate the induction of an extracellular AGLA from Talaromyces flavus. Trehalose, a disaccharide made of two glucose units stimulates several lysosomal hydrolases including the AGLA through autophagy mediated mechanism. Thus, the present invention has found a potent small molecule, which is routinely consumed as a supplement, which can substantially stimulate AGLA activity.

Thew present invention include direct proof that FA can substantially stimulate small molecule chaperone DGJ mediated refolding of AGLA in both, the recombinant AGLA as well as in a cell line from a FD affected individual. DGJ stabilizes the misfolded protein, and helps it refold in proper conformation resulting in partial restoration of enzyme function. FA incubation further stimulates the refolded enzyme thereby augmenting AGLA enzymatic activity. The lymphoblastoid cell line tested was from an individual with confirmed FD diagnosis. This individual is a male and was 41 years old at the time of establishing the cell line. Genetic analysis revealed him to be hemizygous for an A>G transition in exon 5 of the GLA gene (codon AAT>AGT) resulting in Asn215Ser substitution in the protein. The individual had residual AGLA activity in fibroblasts and WBC and displayed mild clinical manifestations. Because FA is stimulating the pure AGLA activity, its inclusion in small molecule chaperone DGJ-based therapy may also nullify any inhibition the AGLA may encounter by the presence of DGJ.

Thus, stimulation of AGLA by FA supplementation may offer benefits to individuals with FD on either ERT or chaperone therapy. The present invention has shown the potential that FD individuals with residual AGLA activity may be able to augment the enzyme activity further through FA supplementation with likely alleviation in the symptomology. Obviously, this can be confirmed through clinical trials in individuals with FD. Regardless, the present invention has shown that FA supplementation further augments AGLA activity restored by the chaperone DGJ in lymphoblastoid cells from FD individuals, and in pure recombinant enzyme.

What is claimed is:

1. A method of treating a patient with Fabry disease, comprising the step of administering a therapeutic amount of folic acid.

2. The method of claim 1, wherein the step of administering a therapeutic amount of folic acid is performed along with the step of administering a therapeutic amount of an enzyme replacement therapy.

3. The method of claim 2, wherein the enzyme replacement therapy comprises a therapeutic amount of α-Galactosidase A.

4. The method of claim 3, wherein the therapeutic amount of folic acid comprises between 0.4 and 4 milligrams.

5. The method of claim 4, wherein the therapeutic amount of folic acid is administered orally.

6. The method of claim 5, wherein the therapeutic amount of folic acid is administered daily.

7. The method of claim 1, wherein the step of administering a therapeutic amount of folic acid is performed along with the step of administering a therapeutic amount of a chaperone therapy.

8. The method of claim 7, wherein the chaperone therapy comprises a therapeutic amount of 1-deoxygalactonojiromycin.

9. The method of claim 8, wherein the therapeutic amount of folic acid comprises between 0.4 and 4 milligrams.

10. The method of claim 9, wherein the therapeutic amount of folic acid is administered orally.

11. The method of claim 10, wherein the therapeutic amount of folic acid is administered daily.

12. A composition for the treatment of Fabry disease, comprising a therapeutic amount of folic acid in combination with a therapeutic amount of an enzyme replacement therapy.

13. The composition of claim 12, wherein the enzyme replacement therapy comprises a therapeutic amount of α-Galactosidase A.

14. The composition of claim 13, wherein the therapeutic amount of folic acid comprises between 0.4 and 4 milligrams.

15. A composition for the treatment of Fabry disease, comprising a therapeutic amount of folic acid in combination with a therapeutic amount of a chaperone therapy.

16. The composition of claim 15, wherein the chaperone therapy comprises a therapeutic amount of 1-deoxygalactonojiromycin.

17. The composition of claim 16, wherein the therapeutic amount of folic acid comprises between 0.4 and 4 milligrams.
